(19) United States
(12) Patent Application Publication
(10) Pub. No.: US 2017/0294612 A1
(43) Pub. Date: Oct. 12, 2017

(54) COMPOSITE CONTAINING SILVER NANOPARTICLES AND ANTIBACTERIAL AGENT, PHOTOELECTRIC CONVERTER, PHOTOSENSITIVE POINTING DEVICE, AND THIN-FILM PHOTOVOLTAIC CELL USING THIS COMPOSITE

(71) Applicants: Nishimatsu Construction Co., Ltd., Tokyo (JP); NONPROFIT ORGANIZATION ITO LABORATORY, Fukuoka-shi, Fukuoka (JP)

(72) Inventors: Kengo ITO, Kanagawa (JP); Katsuya IWANAGA, Tokyo (JP)

(73) Assignees: Nishimatsu Construction Co., Ltd., Tokyo (JP); Ito Research Institute Co., Ltd., Fujisawa-shi, Kanagawa (JP)

(21) Appl. No.: 15/507,900

(22) PCT Filed: Sep. 5, 2014
(86) PCT No.: PCT/JP2014/004567

§ 371 Date: Mar. 1, 2017

(51) Int. Cl. H01L 51/42; H01L 31/0445; H01L 51/00

(52) U.S. Cl. CPC H01L 51/426; H01L 51/0051; H01L 51/008; H01L 51/0094; H01L 31/0445

(57) ABSTRACT

[Problem] The purpose of the present invention is to provide a novel optical functional material in which silver nanoparticles are used. [Solution] According to the present invention, a ternary composite formed by mixing silver nanoparticles, an organic semiconductor, and a clay in a liquid phase is provided. The organic semiconductor is preferably an organic charge-transfer complex, and more preferably a charge-transfer boron polymer. The clay is a layered silicate mineral, and preferably smectite. The present invention also provides an antibacterial agent, a photoelectric converter, and a photosensitive pointing device using the ternary composite.

### TECHNICAL FIELD

The present invention relates to an optically functional material. More particularly, the present invention relates to an optically functional material that makes use of silver nanoparticles and to applications thereof.

### BACKGROUND ART

Conventionally, titanium oxide (TiO₂) has been studied from various angles for its use as an antibacterial agent because of its photocatalytic effect (for example, Patent Document 1). Titanium oxide, however, exhibits a photocatalytic effect only in the presence of ultraviolet light, and thus is incapable of performing an antibacterial treatment inside a room where outdoor light does not enter, such as an operating room.

Meanwhile, silver particles of nano-scale order (silver nanoparticles) are known to absorb strong light incident on them through localized surface plasmon resonance (LSPR).

### PRIOR ART DOCUMENT

### Patent Document

Patent Document 1: Japanese Unexamined Patent Application Publication No. H11-169727

### SUMMARY OF THE INVENTION

### Problem to be Solved by the Invention

In view of the above-described problems of the conventional art, the present invention has an objective of providing a novel optically functional material that makes use of silver nanoparticles.

### Means for Solving the Problems

The present inventors have gone through an earnest study of a novel optically functional material that makes use of silver nanoparticles, and as a result of which conceived of the following structure and achieved the present invention.

Specifically, the present invention provides a ternary composite that is obtained by mixing silver nanoparticles, an organic semiconductor and a clay in liquid phases. This organic semiconductor is preferably an organic charge-transfer complex, and more preferably a charge-transfer type boron polymer. Moreover, the above-mentioned clay is a layered silicate mineral, and preferably smectite.

Furthermore, the present invention provides an antibacterial agent, a photoelectric conversion element and a photosensitive pointing device that utilize the above-described ternary composite.

In addition, the present invention provides a thin-film solar cell that utilizes a binary composite obtained by mixing silver nanoparticles and a clay in liquid phases.

### Effect of the Invention

As described above, the present invention provides, as a novel optically functional material, a composite containing silver nanoparticles, and provides an antibacterial agent, a photoelectric conversion element, a photosensitive pointing device and a thin-film solar cell, which utilize said composite.

### BRIEF DESCRIPTION OF DRAWINGS

FIG. 1 A diagram showing steps for producing a ternary composite of an embodiment of the present invention.

FIG. 2 A conceptual view for illustrating a method for preparing a raw material liquid A (silver nanoparticle aqueous dispersion).

FIG. 3 Diagrams showing a charge-transfer type boron polymer as a material of a raw material liquid B.

FIG. 4 Schematic views of photoelectric conversion elements according to an embodiment of the present invention.

FIG. 5 A schematic view of a photosensitive-type pointing device according to an embodiment of the present invention.

FIG. 6 A schematic view of a thin-film solar cell according to an embodiment of the present invention.

FIG. 7 A diagram showing an absorption spectrum of the raw material liquid A (silver nanoparticle aqueous dispersion).

FIG. 8 A view showing an experimental cell.

FIG. 9 A diagram showing results from measurement of photovoltaic power.

FIG. 10 A view showing an experimental cell.

FIG. 11 A diagram showing results from measurement of current.

FIG. 12 A diagram showing an absorption spectrum of the raw material liquid A (silver nanoparticle aqueous dispersion).

### BEST MODES FOR CARRYING OUT THE INVENTION

Hereinafter, the present invention will be described by way of embodiments shown in the drawings, although the present invention should not be limited to the embodiments shown in the drawings.

### First Embodiment

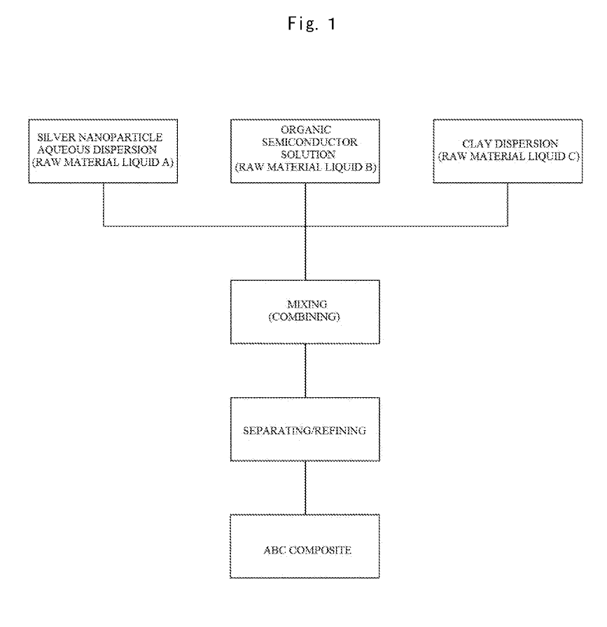

A ternary composite as a first embodiment of the present invention is an optically functional material obtained by mixing silver nanoparticles, an organic semiconductor and a clay in liquid phases. Hereinafter, a method for producing the ternary composite will be described with reference to FIG. 1.

(Preparation of Raw Material Liquid A: Silver Nanoparticle Aqueous Dispersion)

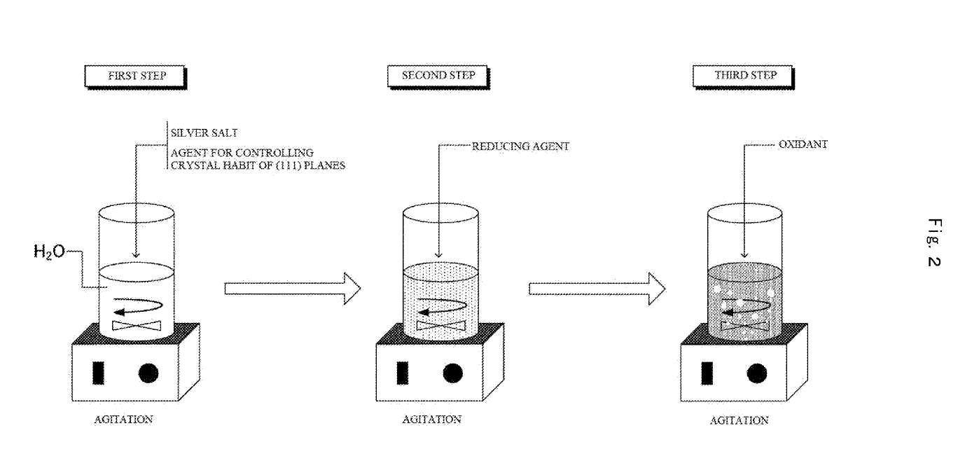

A silver nanoparticle aqueous dispersion is prepared by a liquid phase reduction method. While the photosensitive wavelength region of the ternary composite as the final product depends on the absorption wavelength region resulting from the localized surface plasmon resonance of the silver nanoparticles contained in the raw material liquid A, the absorption wavelength region of the plasmon resonance is known to depend on the crystalline size of the silver nanoparticles. In this regard, according to the following method, silver nanoparticles that have an absorption wavelength region of the plasmon resonance from a visible region to an infrared region can be produced well under control. Hereinafter, a preferable method for preparing the raw material liquid A will be described with reference to FIG. 2.

As shown in FIG. 2, the preparation method of the present embodiment comprises basically three steps. First, in the first step, a aqueous silver ion solution containing a crystal habit controlling agent is prepared. Specifically, silver salt such as silver nitrate (AgNO₃) and a crystal habit controlling agent are added to water (preferably pure water, more preferably ultrapure water) under vigorous agitation to prepare an aqueous silver ion solution. Here, a preferable example of the crystal habit controlling agent used in the present embodiment include citric acid that has a selective adsorptive property to (111) planes of silver crystals.

In the subsequent second step, a reducing agent is added to the above-described aqueous silver ion solution under vigorous agitation. The added reducing agent reduces silver ion in the aqueous solution to form very fine silver crystals. A preferable example of the reducing agent used in the present embodiment includes sodium tetrahydroborate (NaBH₄).

In the subsequent third step, an oxidant is added to the aqueous dispersion containing the fine silver crystals obtained by the above-described procedure under vigorous agitation. A preferable example of the oxidant used in the present embodiment includes hydrogen peroxide (H₂O₂). Once the oxidant is added, the solubility of the metal silver in the aqueous dispersion increases such that some of the fine silver crystals ionize again. Accordingly, the oxidant is added for a plurality of times or the oxidant is continuously added while controlling the additive flow rate so as to allow a constant level of silver ion to consistently and stably be present in the reaction system, as a result of which Ostwald ripening proceeds where larger crystals are selectively grown while smaller crystals vanish. Consequently, plate-like silver nanoparticles with principal planes having elongated major axes remain as the main component in the reaction system. The thus-obtained silver nanoparticles with larger size have the absorption wavelength region of the plasmon resonance from the visible region to the infrared region.

While it is necessary to control the size of the silver nanoparticles of the raw material liquid A according to the photosensitive wavelength region expected for the ternary composite as the final product, the size of the silver nanoparticles can be controlled in the present embodiment by adjusting the concentrations of the silver ion and the crystal habit controlling agent in the first step, as well as the amount of the reducing agent added, the agitation efficiency, the reaction temperature and the like in the second step.

(Preparation of Raw Material Liquid B: Organic Semiconductor Solution)

An organic semiconductor is added to a suitable organic solvent, and the resultant is subjected to mixing/agitating to prepare an organic solution of the organic semiconductor. An “organic semiconductor” as used herein refers to an organic substance that exhibits the nature of a semiconductor, which is preferably an organic charge-transfer complex and more preferably a charge-transfer type boron polymer having a nitrogen atom-boron atom complex structure.

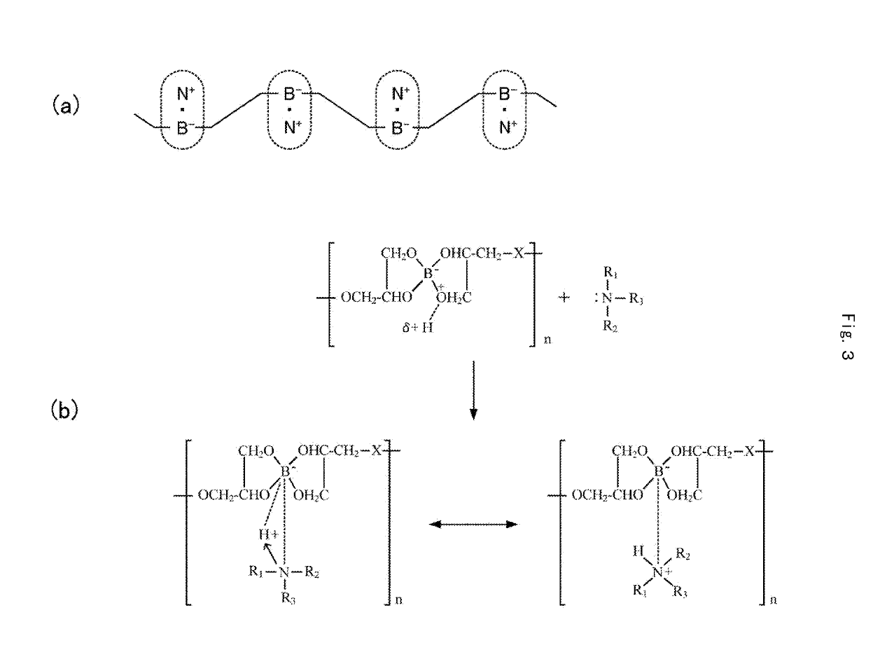

FIG. 3(a) schematically shows a molecular structure of a charge-transfer type boron polymer that is preferable as a material for a raw material liquid B. Here, a charge-transfer type boron polymer is a polymeric charge-transfer type conjugate that can be obtained by reacting a semipolar organic boron polymer compound with a tertiary amine. As shown in FIG. 3(b), in the charge-transfer type boron polymer, the semipolar bond moiety of the semipolar organic boron polymer binds to the basic nitrogen bind to form an ion pair. The resulting acidic protons migrate while leaving the binding properties to both boron and nitrogen sides, thereby presenting a resonance structure. This is considered to allow the electrons to move and result the Fermi level that is responsible for the behavior as a p-type semiconductor. Here, the charge-transfer type boron polymer represented by the structural formula shown in FIG. 3(b) is merely an example, and it is needless to say that the material of the raw material liquid B is not limited thereto.

(Preparation of Raw Material Liquid C: Clay Dispersion)

A clay is added to a suitable organic solvent and the resultant is subjected to mixing/agitating to prepare an organic dispersion of the clay. A “clay” as used herein refers to a layered silicate mineral, and preferably smectite. In the present embodiment, a clay that has been lipophilized by replacing an interlaminar cation with an organic ion is preferably used.

(Three-Liquid Mixing)

Finally, the raw material liquids A, B and C prepared by the above-described procedures are mixed/agitated at a suitable blending ratio, and then left to stand for a sufficient period of time. Meanwhile, the silver nanoparticles, the clay and the organic semiconductor in the mixed solution electrostatically adsorb to and combine with each other, thereby obtaining an ABC composite. Hereinafter, this ternary composite is referred to as an ABC composite. According to the present embodiment, an ABC composite formed in a mixed solution is separated by a suitable method, and refined by a method according to the intended use.

As a method for producing an ABC composite has been described heretofore, an internal photoelectric effect of the ABC composite as an optically functional material will be described hereinbelow. The present inventors presume the structure of the ABC composite and the mechanism of the internal photoelectric effect thereof as follows.

The present inventors presume that the organic semiconductor molecules are adsorbed on the surfaces of the silver nanoparticles in the ABC composite, and that a built-in potential difference due to Schottky junction is caused on the organic semiconductor side in the vicinity of the junction interface between the organic semiconductor molecules and the particles.

When light is incident on the ABC composite, free electrons of the organic semiconductor in the vicinity of the junction interface with the silver nanoparticles are excited. While excitation with this light energy alone does not allow the free electrons of the organic semiconductor to exceed the band gap, it is presumed that further excitation due to electric field enhancement by the localized surface plasmon resonance generated in the silver nanoparticles allows the free electrons of the organic semiconductor to exceed the band gap and result in carrier separation (photoinduced charge separation).

In the ABC composite, it is presumed that the clay molecule plays a role of aligning the orientation of the organic semiconductor molecule by adsorbing, on its interlaminar, a plurality of organic semiconductor molecules in a bundle, thereby enhancing the conductivity of the organic semiconductor molecule.

While the internal photoelectric effect of the ABC composite has been described heretofore, applications of the ABC composite as an optically functional material will be described hereinbelow.

(Application as Antibacterial Agent)

The ABC composite can be applied to an antibacterial agent. An antibacterial agent of the present embodiment expresses an antibacterial activity upon receiving light. The present inventors presume the mechanism of this expression of the antibacterial activity in response to light as follows.

First, it is considered that charge of 1 volt or more is maintained at the interface with the air due to photoinduced charge separation caused in the ABC composite, so that bacteria, viruses or the like in the air can be killed through the so-called electric field sterilization action. Secondly, it is considered that, by the same principle as titanium oxide, carriers caused by photoinduced charge separation oxidize/reduce water in the air to produce active oxygen species, which decompose bacteria, viruses or the like in the air.

While titanium oxide does not exert an antibacterial action in a dark place, the antibacterial agent of the present embodiment exerts an antibacterial action in a dark place as well since the silver nanoparticles, boron contained in the charge-transfer type boron polymer and the clay, i.e., the structural components of the ABC composite as an antibacterial component, each independently have an antibacterial property.

Moreover, while a sensitive wavelength region of titanium oxide is limited to an ultraviolet region, the sensitive wavelength region of the antibacterial agent of the present embodiment can arbitrarily be set by controlling the absorption wavelength region of the plasmon resonance absorption of the silver nanoparticles configuring the ABC composite, i.e., the antibacterial component (that is, by controlling the crystalline size of the silver nanoparticles). For example, the sensitive wavelength region of the antibacterial agent may be set to include the wavelength region of illuminating light (visible region) so that it can express a strong antibacterial activity inside a room where outdoor light does not enter, such as an operating room. While the problem of multidrug-resistant bacteria at medical facilities is becoming serious in recent years, the antibacterial agent of the present embodiment can be expected to be of some help to solve this problem. In addition, by setting the sensitive wavelength region of the antibacterial agent to include a wavelength region of an infrared region, a strong antibacterial activity can be expressed outdoor by utilizing the enormous amount of infrared radiation energy from sun light.

(Application as Photoelectric Conversion Element)

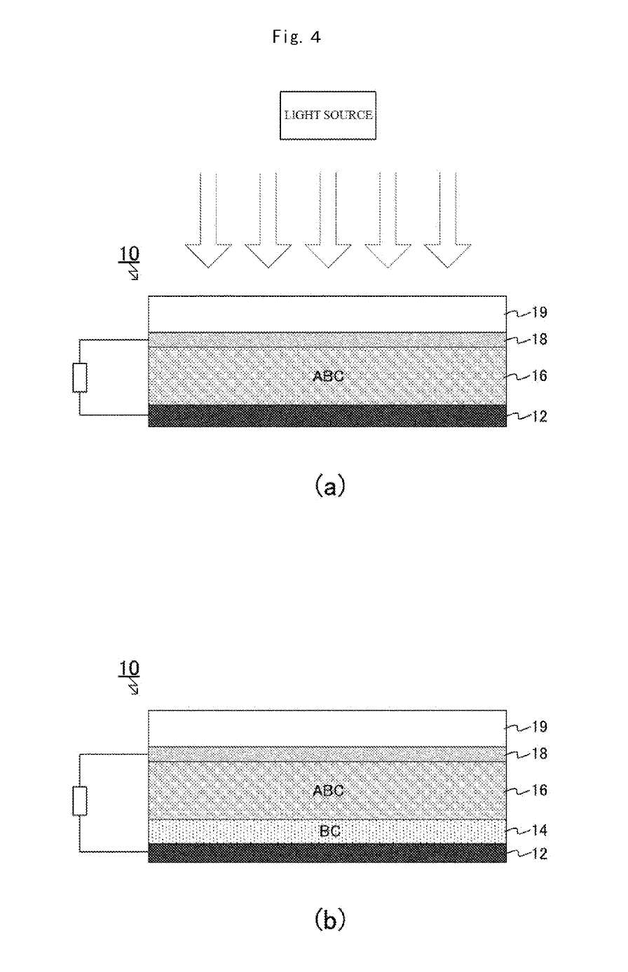

The ABC composite can be applied to a photoelectric conversion element. A photoelectric conversion element as used herein includes optical sensors and solar cells. FIG. 4 shows schematic views of a photoelectric conversion elements 10 incorporating the ABC composite. As shown in FIG. 4(a), the photoelectric conversion element 10 is provided with a structure in which an ABC composite layer 16 including the ABC composite, a transparent electrode layer 18 (ITO, SnO₂, etc.) and a transparent substrate 19 (glass, plastic, etc.) are laminated on a rear electrode 12 (Al, etc.), where the ABC composite layer 16 serves as a photoelectric conversion layer.

When the photoelectric conversion element 10 of the present embodiment is irradiated with light, the light passes through the transparent substrate 19 and the transparent electrode layer 18 and enters the ABC composite layer 16. Upon this, photoinduced charge separation occurs in the vicinity of the junction interface between the silver nanoparticles and the organic semiconductor forming the ABC composite. Free electrons that exceed the band gap move to the transparent electrode layer 18 side via the silver nanoparticles in the ABC composite layer 16 while holes move to the rear electrode 12 side via the organic semiconductor in the ABC composite layer, as a result of which current flows between both electrodes.

Furthermore, the photoelectric conversion layer of the photoelectric conversion element 10 may have a two-layer structure that has an ABC composite layer 16 and a BC composite layer 14 laminated as shown in FIG. 4(b). Here, the BC composite is a binary composite that is obtained by mixing an organic semiconductor and a clay, i.e., the structural components of the ABC composite, in liquid phases. In this case, the direction of the current is stabilized due to the difference in the work functions between the ABC composite layer 16 and the BC composite layer 14. The ABC composite layer and the BC composite layer may be formed by a transfer method or a film formation method by coating (the also same applies hereinafter).

In the photoelectric conversion element 10 of the present embodiment, the crystalline size of the silver nanoparticles configuring the ABC composite can be controlled to arbitrarily set the sensitive wavelength region thereof. Accordingly, by setting the sensitive wavelength region of the ABC composite to include an infrared region, electricity can be taken out from the enormous amount of infrared radiation energy of sun light that could not be utilized hitherto.

(Application as Photosensitive-Type Pointing Device)

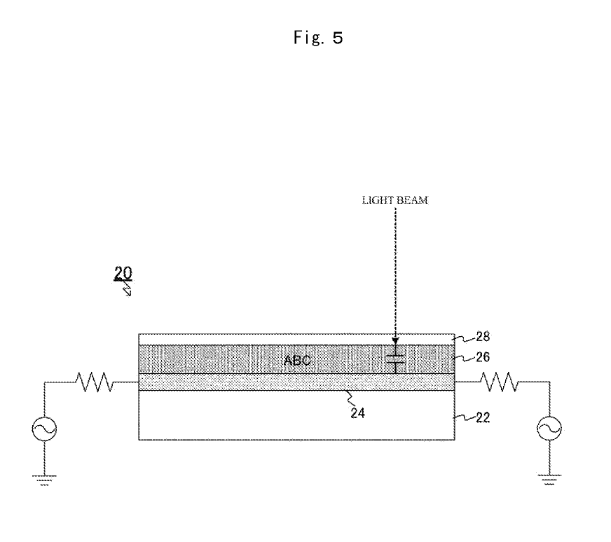

The ABC composite can be applied to a photosensitive-type pointing device for detecting an incident position of a light beam as an input position. FIG. 5 shows a schematic view of a photosensitive-type pointing device 20 incorporating the ABC composite. As shown in FIG. 5, the photosensitive-type pointing device 20 comprises: a structure in which a transparent electrode layer 24 (ITO, SnO₂, etc.), an ABC composite layer 26 including the ABC composite and a protective layer 28 (glass, plastic, etc.) are laminated on a glass substrate 22; and a position detecting means (not shown) for detecting an input position by an electrostatic capacitance system by applying common-mode alternating-current voltage of the same potential to both sides of the transparent electrode layer 24 via current detecting resistors.

In other words, the photosensitive-type pointing device 20 of the present embodiment has a structure which is obtained by inserting a layer including the ABC composite between a transparent electrode layer and a protective layer of a conventional electrostatic-capacitance-type touch panel structure.

In the photosensitive-type pointing device 20 of the present embodiment, the crystalline size of the silver nanoparticles configuring the ABC composite can be controlled so as to arbitrarily set the sensitive wavelength region thereof.

While a conventional electrostatic-capacitance-type touch panel detects a position touched by a finger as an input position, the photosensitive-type pointing device 20 of the present embodiment detects an incident position of a light beam as an input position. As shown in FIG. 5, when a light beam of a predetermined wavelength is irradiated on the photosensitive-type pointing device 20, this light beam passes through the protective layer 28 and enters the ABC composite layer 26. This causes a change in the electrostatic capacitance due to photoinduced charge separation at the site of the ABC composite layer 26 where the light beam is incident, and this change is detected by the position detecting means (not shown).

While a ternary composite as the first embodiment of the present invention has been described heretofore, a binary composite as a second embodiment of the present invention will be described hereinbelow.

### Second Embodiment

A binary composite as a second embodiment of the present invention is an optically functional material obtained by mixing silver nanoparticles and a clay in liquid phases. In order to produce the binary composite of the present embodiment, a silver nanoparticle aqueous dispersion (raw material liquid A) and a clay dispersion (raw material liquid C) are mixed/agitated at a suitable blending ratio, and then left to stand for a sufficient period of time. During this period, the silver nanoparticles and the clay in the mixed solution electrostatically adsorb to and combine with each other. Thereafter, this composite is separated/refined by a suitable method to obtain a binary composite of the present embodiment. Hereinafter, this binary composite is referred to as an AC composite. The methods for preparing the raw material liquid A and the raw material liquid C are basically the same as those described in the first embodiment. Preferably, the raw material liquid C is prepared using a hydrophilic clay (preferably, smectite).

While a method for producing an AC composite has been described heretofore, applications of the AC composite as an optically functional material will be described hereinbelow.

(Application as Functional Layer for Enhancing Power Generation Efficiency of Thin-Film Solar Cell)

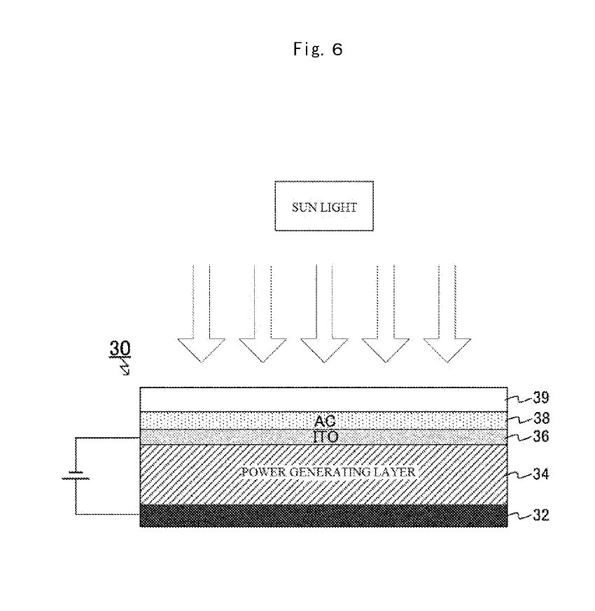

The AC composite can be applied to a functional layer for enhancing a power generation efficiency of a thin-film solar cell. FIG. 6 shows a schematic view of a thin-film solar cell 30 which incorporates the AC composite. As shown in FIG. 6, the thin-film solar cell 30 is provided with a structure in which a power generating layer 34 (an organic semiconductor compound, a CIGS compound, an amorphous silicon, etc.), an ITO transparent electrode layer 36, an AC composite layer 38 including the AC composite and a transparent substrate 39 (glass, plastic, etc.) are laminated on a rear electrode 32 (Al, etc.).

In other words, the thin-film solar cell 30 of the present embodiment has a structure which is obtained by inserting a layer including the AC composite between an ITO transparent electrode layer coating a power generating layer and a transparent substrate, in a structure of a conventional thin-film solar cell. The present inventors presume that employment of this structure allows enhancement of the power generation efficiency of the thin-film solar cell because of the following reasons.

As shown in FIG. 6, as sun light is incident on the thin-film solar cell 30, the sun light passes through the transparent substrate 39, the AC composite layer 38 and the ITO transparent electrode layer 36, and reaches the power generating layer 34 where electromotive force is generated due to photoinduced charge separation. Upon this, the sun light passing through the AC composite layer 38 causes localized surface plasmon resonance on the surfaces of the silver nanoparticles configuring the AC composite. As a result, photoinduced charge separation is caused in the vicinity of the junction interface between the silver nanoparticles and ITO (semiconductor) of the transparent electrode layer 36. The power generation efficiency of the thin-film solar cell 30 is presumably enhanced as the electromotive force generated in the ITO and the electromotive force generated in the power generating layer 34 are superimposed.

In the thin-film solar cell 30 of the present embodiment, the crystalline size of the silver nanoparticles configuring the AC composite can be controlled to arbitrarily set the sensitive wavelength region thereof. Accordingly, by setting the sensitive wavelength region of the AC composite to include an infrared region, electricity can be taken out from the enormous amount of infrared radiation energy of sun light that could not be utilized hitherto.

Thus, as described above, the present invention is capable of providing a composite that contains silver nanoparticles, as a novel optically functional material. The composite of the present invention can easily be produced by a wet process at normal temperature and pressure, and can easily be made to have a larger plane area, while its sensitive wavelength region can arbitrarily be set from an ultraviolet region to an infrared region. Thus, it is expected to be widely applicable and developable as an optically functional material.

So far, the present invention has been described by way of embodiments, although the present invention should not be limited to the above-described embodiments. Any other embodiments that could be conceived by those skilled in the art are within the scope of the present invention as long as they have the action/effect of the present invention.

### EXAMPLES

Hereinafter, an ABC composite of the present invention will be described specifically by way of examples, although the present invention should not be limited to the following examples.

<Preparation of ABC Composite>

An ABC composite of the present invention was prepared according to the following procedure. Here, all of the reagents used were those of special grades from Wako Pure Chemical Industries.

(Preparation of Raw Material Liquid A: Silver Nanoparticle Aqueous Dispersion)

60 mL of a 500 mM aqueous trisodium citrate solution and 20 mL of a 100 mM silver nitrate aqueous solution were sequentially added to 10 liters of pure water under agitation to prepare a first liquid. Additionally, 0.76 g of sodium tetrahydroborate was added and dissolved in 10 liters of ultrapure water under agitation to prepare a second liquid. Subsequently, each of the first and second liquids was sent to a static mixer to be mixed at a flow rate of 5 liters/minute using a diaphragm pump. Consequently, the mixed liquid presented pale yellow color.

407 μl of 300 mM aqueous trisodium citrate solution was added to the mixed liquid (1650 mL) presenting pale yellow color under agitation, to which 990 μl of 30% hydrogen peroxide water was further added and agitated for 3 hours. Consequently, a silver nanoparticle aqueous dispersion (0.001 wt %) presenting indigo blue was obtained.

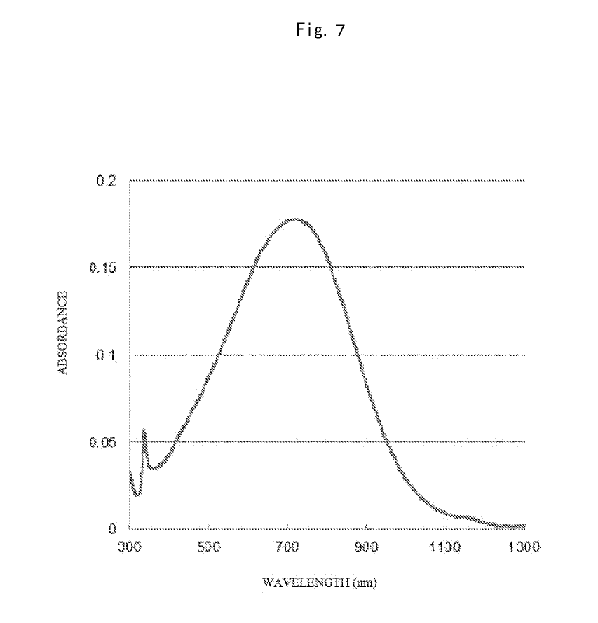

FIG. 7 shows results from measurement of the absorption spectrum of the silver nanoparticle aqueous dispersion prepared according to the above-described procedure with a spectrophotometer (V-670 UV/Vis/NIR, manufactured by JASCO Corporation). As can be appreciated from FIG. 7, a sharp absorption band corresponding to plate-like silver nanoparticles appears around 336 nm while a broad absorption band with a peak around 720 nm appeared. Meanwhile, an absorption band corresponding to non-plate-like silver nanoparticles (a band with a peak around 400 to 420 nm) did not appear. From these results, the prepared silver nanoparticle aqueous dispersion was shown to contain generally only plate-like silver nanoparticles.

(Preparation of Raw Material Liquid B: Organic Semiconductor Solution)

An antistatic agent (BN-2, manufactured by Boron International) was used as the organic semiconductor. 1 g of BN-2 was weighed and placed into a 300-mL beaker, to which 100 g of ethanol was added and the resultant was subjected to ultrasonic irradiation, thereby obtaining a BN-2 solution (1 wt % ethanol).

(Preparation of Raw Material Liquid C: Clay Dispersion)

Lipophilic synthetic smectite (SAN, manufactured by Co-op Chemical) was used as the clay. 1 g of white SAN powder was weighed and placed into a 300-mL beaker, to which 100 g of toluene was added and the resultant was subjected to ultrasonic irradiation for 15 minutes, thereby obtaining a clay dispersion (2 wt % toluene).

(Mixing of Three Liquids)

The raw material liquids A to C were mixed such that the solid content ratio of the silver nanoparticles, BN-2 and SAN was 1:2:1. Specifically, the raw material liquid B (972 μl) and the raw material liquid C (243 μl) were added to and mixed with 15 mL of butyl acetate, to which the raw material liquid A (486 mL) was loaded while agitating at high speed with a stirrer. After several minutes of agitation, the resultant was left to stand overnight. As a result, the liquid in the container phase-separated into a colorless water layer (lower layer) and a butyl acetate layer (upper layer), with a dark navy layer appearing at the interface therebetween. This navy layer was collected into a test tube and the solvent was distilled away, thereby obtaining a paste-like ABC composite.

<Verification of Antibacterial Activity>

The antibacterial activity of the ABC composite of the present invention was examined according to the following procedure.

(Preparation of Bacteria Solution)

As a test bacterium, enteropathogenic Escherichia coli, E. coli 0157:H7, was used. Specifically, a strain of E. coli 0157:H7 uniquely separated by the Fukuoka Institute of Health and Environmental Sciences was refreshed and then used for inoculation to give 10 mL of a stock liquid medium, which was cultured in a shaker (30° C./24 hours/120 rpm). This culture solution was diluted 10⁷-fold to be used a bacteria solution.

(Culture Conditions)

The paste-like ABC composite prepared according to the above-described procedure was diluted 128-fold with butyl acetate. Hereinafter, this 128-fold dilution is referred to as the antibacterial agent. Next, stock plate media (1/100 concentration) were prepared to inoculate a plate medium applied with an antibacterial agent and a plate medium without the antibacterial agent with 100 μl of the bacteria solution prepared according to the above-described procedure. Thereafter, the resultants were cultured for several days in an incubator (temperature 30° C.) under the five types of light conditions (1) to (4) indicated below.

(1) Dark place

(2) Irradiation with white light

(3) Irradiation with red light

(4) Irradiation with blue light

(5) Irradiation with green light

Specifically, in this experiment, two media were prepared and cultured for each of 10 types of combinations, i.e., the presence or the absence of the antibacterial agent and the five types of light conditions. Moreover, LED was used as the light source, where the intensity of irradiation light was 15 μmol.m⁻².s⁻¹, which was equivalent to indoor light level.

(Culture Results)

After several days of culture, the numbers of colonies generated in the media were counted. In this experiment, the average count of the two media prepared for each condition was assumed as the number of colonies of said condition, and the antibacterial rate (%) was determined for each of the five types of light conditions based on Formula (1) below.

$$\begin{matrix}[ Numerical Formula 1 ] & \\ Antibacterial rate ( \% ) = ( 1 - \frac{Number of colonies ( in presence of antibacterial agent )}{Number of colonies ( in absence of antibacterial agent )} ) \times 100 & ( 1 )\end{matrix}$$

The numbers of colonies and the antibacterial rates (%) under the respective conditions are summarized in Table 1 below.

TABLE 1

| | | Dark | White | Red | Blue | Green |
|---|---|---|---|---|---|---|
| | | place | light | light | light | light |
| Number | In presence of | 59 | 41 | 59 | 56 | 71 |
| of | antibacterial | | | | | |
| colonies | agent | | | | | |
| | In absence of | 21 | 5 | 0 | 6 | 7 |
| | antibacterial | | | | | |
| | agent | | | | | |

| | | | | | |
|---|---|---|---|---|---|
| Antibacterial rate (%) | 35.6 | 87.8 | 100 | 89.3 | 90.1 |

From the results shown in Table 1 above, the ABC composite of the present invention showed a certain antibacterial activity in a dark place, where the antibacterial activity was largely enhanced upon receiving light with a wavelength that matched the absorption band of the silver nanoparticles configuring the ABC composite.

<Verification of Internal Photoelectric Effect>

The internal photoelectric effect of the ABC composite of the present invention was examined.

(Preparation of Experimental Cell)

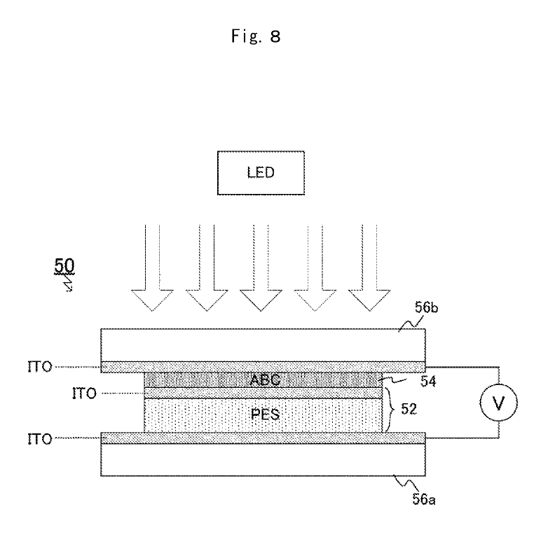

An experimental cell shown in FIG. 8 was prepared according to the following procedure. The above-described navy layer (ABC composite) that appeared at the interface after mixing and phase separating the raw material liquids A to C was transferred onto an ITO surface of a transparent conductive film 52 (thickness of about 200 μm, manufactured by Peccell Technologies) that was obtained by coating a polyether sulphone film (PES) with ITO. The resultant was naturally dried and strongly heated with a drier for 5 minutes to form an ABC composite layer 54 of about 0.2 μm. Subsequently, the transparent conductive film having the ABC composite layer 54 formed thereon was interposed between two glass substrates with ITO electrodes 56a and 56b, and secured with a spring clip, thereby obtaining an experimental cell 50.

(Measurement of Photovoltaic Power)

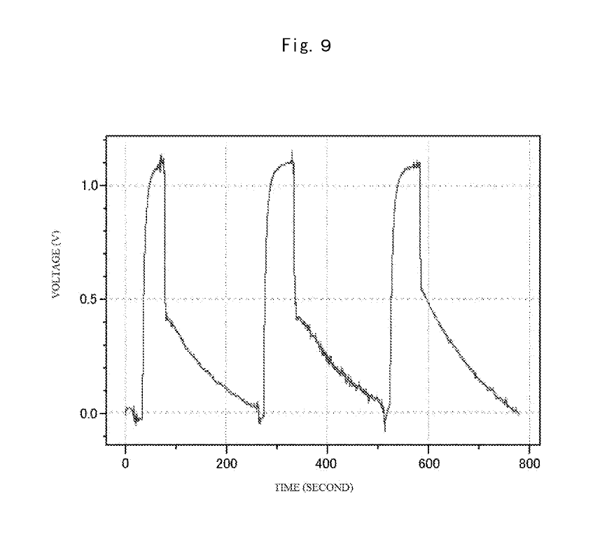

While connecting the leads of the ITO electrodes of the two glass substrates 56 of the experimental cell 50 to a potentiostat/galvanostat (manufactured by IVIUM), a white LED light source was placed on the ABC composite layer 54 side to repeatedly turn on and off the lighting. In this experiment, the white LED light source was placed such that the illuminance on the irradiated area of the experimental cell was 10⁴ lux, and electromotive force generated between the ITO electrodes with on/off of the light source was measured with time. FIG. 9 shows the results from measurement of the photovoltaic power.

As shown in FIG. 9, once the light source was turned on, the electromotive force reached 1.0V within several seconds. From this result, the ABC composite of the present invention was shown to have a photovoltaic power effect. On the other hand, once the light source was turned off, the potential immediately dropped to about 0.5V, and slowly attenuated thereafter.

<Application as Photoelectric Conversion Element>

A photoelectric conversion element including the ABC composite of the present invention was prepared to verify the operation thereof.

(Preparation of Photoelectric Conversion Element)

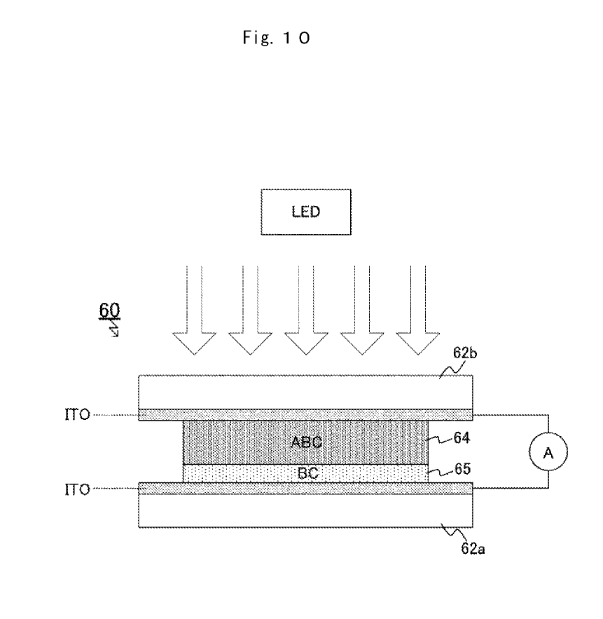

An experiment cell shown in FIG. 10 was prepared according to the following procedure. A silver nitrate aqueous solution obtained by dissolving 54 mg of AgNO₃ into 300 mL of water was refluxed under deaeration to boil. To this, 6 mL of 10 wt % aqueous trisodium citrate solution that has been deaerated for 15 minutes was added, refluxed for about an hour, and left to stand overnight. As a result, a silver nanoparticle aqueous dispersion presenting yellowish gray was obtained. When the absorption spectrum of this silver nanoparticle aqueous dispersion was measured, an absorption band appeared in the vicinity of 410 nm.

When 30 mL of the silver nanoparticle aqueous dispersion prepared according to the above-described procedure was added to 2.5 mL of 1 wt % acetone solution of lipophilic synthetic smectite (STN, manufactured by Co-op Chemical), a sediment exhibiting greenish brown was precipitated. After washing this sediment with methanol and dried at room temperature, the resultant was subjected to ultrasonic dispersion with γ-butyrolactone. As a result, a transparent dispersion presenting green was obtained.

Glass fiber paper with a thickness of 30 μm (manufactured by Nippon Sheet Glass) was immersed in the resulting green dispersion (solid content 3 wt %) and taken out. Next, it was impregnated with an ethanol solution of an antistatic agent (BN-2, manufactured by Boron International) (10 wt %) for 5 minutes. Thereafter, the glass fiber paper was washed with a large amount of methanol and air-dried, thereby obtaining glass fiber paper 64 (dark green) having the ABC composite formed inside.

Next, an ethanol solution (solid content 5 wt %) obtained by blending an antistatic agent (BN-2, manufactured by Boron International) and lipophilic synthetic smectite (SEN, manufactured by Co-op Chemical) at a solid content ratio of 2:1 was applied and dried on the ITO surface of the glass substrate with an ITO electrode 62a to form a BC composite layer 65 having a thickness of about several μm. Subsequently, the glass fiber paper 64 having the ABC composite formed thereon was disposed on the BC composite layer 65, and the other glass substrate with ITO electrode 62b was further disposed thereon such that the ITO surface thereof faced the glass fiber paper 64. The two glass substrates with ITO electrodes 62a and 62b were secured with a spring clip to be used as an experiment cell.

(Measurement of Photocurrent)

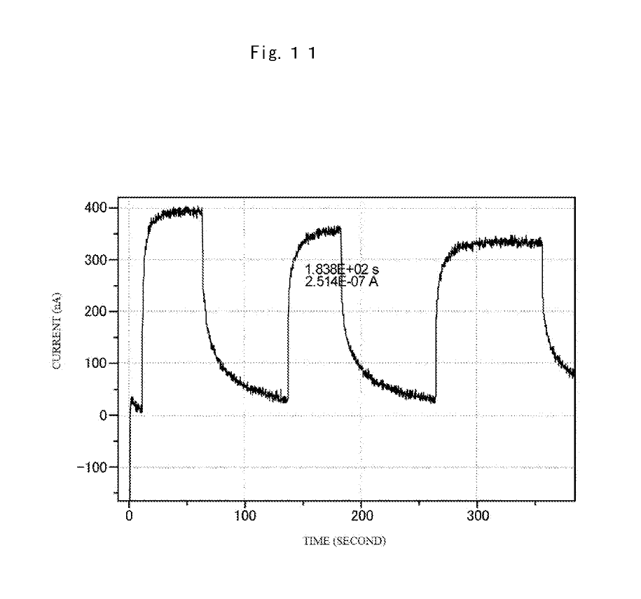

While connecting the leads of the ITO electrodes of the two glass substrates of the prepared experiment cell to a potentiostat/galvanostat (manufactured by IVIUM), a white LED light source was placed on the colored fiber paper side to repeatedly turn on and off the lighting. In this experiment, the white LED light source was placed such that the illuminance on the irradiated area of the experimental cell was 10⁴ lux, and current generated with on/off of the light source was measured with time. FIG. 11 shows the measurement results.

As shown in FIG. 11, once the light source was turned on, the current of about several hundreds of nA rose within several seconds and once the current reached saturation, constant current continuously flowed. From this result, the prepared experiment cell was shown to serve as a photoelectric conversion element. On the other hand, once the light source was turned off, the current value immediately dropped to about several hundreds of nA, and slowly attenuated thereafter.

<Application as Functional Layer for Enhancing Power Generation Efficiency of Thin-Film Solar Cell>

A functional layer composed of an AC composite of the present invention was added to an existing thin-film solar cell to verify the effect thereof.

(Preparation of AC Composite)

2.5 mL of 450 mM aqueous trisodium citrate solution and 750 μl of 100 mM silver nitrate aqueous solution were sequentially added to 140 mL of ultrapure water under agitation to prepare a starting solution. 2.5 mL of 300 mM aqueous sodium tetrahydroborate solution as a reducing agent was added to the prepared starting solution under agitation. Immediately after confirming that the aqueous solution exhibited pale yellow color upon addition of the reducing agent, 3.6 mL of 30% hydrogen peroxide water was added and agitation was continued. Thereafter, the step of adding 3.6 mL of 30% hydrogen peroxide water under agitation was repeated every hour for 14 times. As a result, a light gray silver nanoparticle aqueous dispersion (0.0038 wt %) that diffusely reflects/scatters light well was obtained.

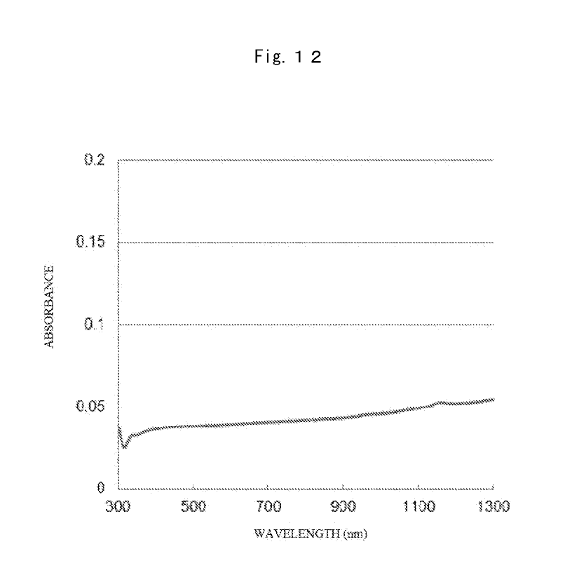

FIG. 12 shows the results from the measurements of the absorption spectra of the silver nanoparticle aqueous dispersion prepared in accordance with the above-described procedure with a spectrophotometer (V-670 UV/Vis/NIR, manufactured by JASCO Corporation). As shown in FIG. 12, a broad absorption band corresponding to plate-like silver nanoparticles appeared in the vicinity of 338 nm. On the other hand, an absorption band corresponding to non-plate-like silver nanoparticles (a band that has a peak in the vicinity of 400 to 420 nm) did not appear. From these results, the prepared silver nanoparticle aqueous dispersion was shown to contain basically only the plate-like silver nanoparticles.

Notable observation of the diffusely reflected/scattered light by naked eyes suggests that the prepared silver nanoparticle aqueous dispersion contains large-sized plate-like particles whose principal planes have major axes reaching micrometer order. Although the absorption band was unable to confirm due to the measurement limit, the prepared silver nanoparticle aqueous dispersion appeared to contain plate-like particles that had a maximum absorption wavelength in an infrared region of 1300 nm or more.

42.5 mL of 1 wt % solution (solvent IPA 75, water 25) of a hydrophilic organified clay (SA#3, manufactured by Kunimine Industries) was added to 100 mL of the silver nanoparticle aqueous dispersion prepared in accordance with the above-described procedure, to which 10 mL of methyl ethyl ketone was further added, agitated and mixed for several minutes using an ultrasonic cleaning machine. As a result, a dispersion of the AC composite of the present invention that presents gray slightly tinged with sky blue was obtained.

A commercially available thin-film solar cell (LL-37, manufactured by PowerFilm) was immersed in isopropyl alcohol a whole day and night and then the laminated layer of the outermost surface was peeled off to expose the ITO transparent electrode layer. Thereafter, the following steps were repeated for several times: the exposed ITO transparent electrode layer was immersed in the AC composite dispersion prepared in accordance with the above-described procedure for several seconds, taken out, immediately blown with air to remove the residual coating and then dried at room temperature with a dryer. As a result, a film of the AC composite was formed on the surface of the ITO transparent electrode layer of the commercially available thin-film solar cell.

(Measurement of Photocurrent)

A thin-film solar cell having the AC composite film formed on the surface of the ITO transparent electrode layer (Example) and a thin-film solar cell without the AC composite film (Comparative example) were prepared. Each of the thin-film solar cell was irradiated with xenon light (wavelength regions: visual to near-infrared) to measure the short-circuit current. As a result, the short-circuit current of the example was found to increase for 10 to 20% as compared to that of the comparative example.

### DESCRIPTION OF REFERENCE NUMERALS

10 . . . Photoelectric conversion element, 12 . . . Rear electrode, 14 . . . BC composite layer, 16 . . . ABC composite layer, 18 . . . Transparent electrode layer, 19 . . . Transparent substrate, 20 . . . Photosensitive-type pointing device, 22 . . . Glass substrate, 24 . . . Transparent electrode layer, 26 . . . ABC composite layer, 28 . . . Protective layer, 30 . . . Thin-film solar cell, 32 . . . Rear electrode, 34 . . . Power generating layer, 36 . . . ITO transparent electrode layer, 38 . . . AC composite layer, 39 . . . Transparent substrate, 50 . . . Experimental cell, 52 . . . Transparent conductive film, 54 . . . ABC composite layer, 56 . . . Glass substrate with ITO electrode, 62 . . . Glass substrate with ITO electrode, 64 . . . Glass fiber paper, 65 . . . BC composite layer

1. A ternary composite obtained by mixing silver nanoparticles, an organic semiconductor and a clay in liquid phases.

2. The ternary composite according to claim 1, wherein the organic semiconductor is an organic charge-transfer complex.

3. The ternary composite according to claim 2, wherein the organic charge-transfer complex is a charge-transfer type boron polymer.

4. The ternary composite according to any one of claims 1 to 3, wherein the clay is a layered silicate mineral.

5. The ternary composite according to claim 4, wherein the layered silicate mineral is smectite.

6. The ternary composite according to claim 1, wherein the silver nanoparticles comprise plate-like particles as a main component.

7. An antibacterial agent comprising the ternary composite according to claim 1 as an antibacterial component.

8. The antibacterial agent according to claim 7, wherein an absorption wavelength region of plasmon resonance absorption of the silver nanoparticles comprises a visible region, and wherein the antibacterial agent expresses an antibacterial activity upon receiving visible light.

9. The antibacterial agent according to claim 7, wherein an absorption wavelength region of plasmon resonance absorption of the silver nanoparticles comprises an infrared region, and wherein the antibacterial agent expresses an antibacterial activity upon receiving infrared light.

10. A photoelectric conversion element comprising a photoelectric conversion layer which is formed with the ternary composite according to claim 1.

11. The photoelectric conversion element according to claim 10, wherein the photoelectric conversion layer is obtained by laminating a layer including the ternary composite and a layer including a binary composite, and wherein the binary composite is obtained by mixing an organic semiconductor and a clay in liquid phases.

12. The photoelectric conversion element according to claim 10 or 11, wherein an absorption wavelength region of plasmon resonance absorption of the silver nanoparticles includes an infrared region, and wherein the photoelectric conversion element converts infrared light into electricity.

13. A photosensitive pointing device comprising:
a transparent electrode, covering a transparent substrate;
a layer including the ternary composite according to claim 1, formed on the transparent electrode; and
a position detecting means for detecting an input position by an electrostatic capacitance system by applying an alternating-current voltage to the transparent electrode,
wherein the position detecting means detects an incident position of a light beam as the input position based on a change in the electrostatic capacitance caused by photoinduced charge separation in the ternary composite.

14. A thin-film solar cell comprising:
an ITO transparent electrode, covering a power generating layer; and
a layer including a binary composite, formed on the transparent electrode,
wherein the binary composite is obtained by mixing silver nanoparticles and a clay in liquid phases.

15. The thin-film solar cell according to claim 14, wherein an absorption wavelength region of plasmon resonance absorption of the silver nanoparticles includes an infrared region, and wherein the thin-film solar cell converts infrared light into electricity.
